(19) United States
(12) Patent Application Publication
(10) Pub. No.: US 2015/0305907 A1
(43) Pub. Date: Oct. 29, 2015

(54) IMPROVED MEDICAL CAST

(71) Applicant: VINCENT MEDICAL MANUFACTURING CO., LTD., Hung Hom (HK)

(72) Inventors: Kwok Fu Fu, Hung Hom (HK); Ching Sau Chu, Hung Hom (HK); Cheuk Tat Wong, Kowloon (HK)

(21) Appl. No.: 14/648,402

(22) PCT Filed: Oct. 15, 2013
(86) PCT No.: PCT/CN2013/085255

§ 371 Date: May 29, 2015

(30) Foreign Application Priority Data
Nov. 29, 2012 (CN) 2012220639809.5

(51) Int. Cl. A61F 5/01

(52) U.S. Cl. CPC A61F 5/012; A61F 5/013

(57) ABSTRACT

A medical cast includes an inflatable air cushion, a foam material within the inflatable air cushion, a closable supporting structure external to the inflatable air cushion, and a sealable air intake connected to the inflatable air cushion. The closable supporting structure is of a size and shape sufficient to enclose a body part.

### PRIORITY INFORMATION

This application claims priority to Chinese Invention Application No. 201220639809.5, filed on Nov. 29, 2012.

### FIELD OF THE INVENTION

The present invention relates to medical casts for immobilizing broken or fractured bones so as to keep the bones aligned and to improve healing. More specifically, the present invention relates to inflatable medical casts for immobilizing broken or fractured bones so as to keep the bones aligned and to improve healing. An external supporting frame may also be included.

### BACKGROUND

Due to aging of the population, a large population with osteoporosis and the lack of knowledge in industrial safety and other issues, humeral shaft fractures have become a high concern among the Chinese medical community. Humeral shaft fracture account for about 5% of all bone fractures, and its natural healing time is about twelve weeks. Currently, when treating patients with bone fractures, the hospital generally uses plaster bandage to fix fractured parts. That is the bandage is impregnated with plaster. The bandage is soaked with water and wrapped around the fractured limb of the patient. Then the plaster bandage will harden after it is air dried naturally so as to fix the fractures.

The abovementioned treatment brings the patient a lot of pain. In particular, plaster is corrosive and may irritate the skin of the limb. It is neither ventilated nor breathable and causes skin erosion. Moreover, the plaster bandage takes a long time to remove and it is difficult to remove. Although sometimes an external supporting frame is also currently used as a fixation method to treat bone fractures, this method still possesses problems. For example, the closed surrounding pressure of the rigid functional external supporting frame is uneven, and the angle cannot be fine-tuned. This reduces its ability to enhance the healing of the humerus. Besides, long-term immobilization of the muscle after fracture leads to muscle atrophy. The pressure of the functional external supporting frame on the atrophied muscle will decrease, or the supporting frame may even become unable to maintain its fixating effect such that the broken bone cannot be united properly. This leads to a greater risk in malunion fractures (mis-alignment).

Thus, broken bones and fractured bones are treated by re-aligning them and then immobilizing them in a cast for a period of time sufficient to allow the bone to knit together and heal. Oftentimes this healing process can take many months as bone growth and repair is relatively slow as compared to muscle growth and repair.

In the cases of long bones such as arm or leg bones this can lead to very large plaster casts which are extremely heavy and unwieldy. Such rigid casts also are typically worn by the patient for upwards of 2 months and thus the muscles that they cover tend to atrophy and shrink as they lose muscle mass. In some cases up to 50% of muscle mass is lost, leading to a large gap between the rigid cast and the arm, leg, etc. As the rigid cast is unable to adjust, the patient is either left with an ill-fitting cast or the physician may in some cases cut away the cast and replace it with a new one.

In addition, the physician may wish to check on the healing progress via X-rays or other scanning techniques. However, due to their density, typical casts may be opaque to or be otherwise incompatible with X-Rays and/or other scanning techniques.

Some inflatable splints have appeared in literature, publications, and in practice but these are typically only used by first-responders at the injury scene and then replaced with a plaster cast at the hospital or final treatment site. Other inflatable casts are known which merely contain inflatable bags, sometimes surrounded by foam or cloth to reduce abrasion to the patient. However, it is found that such foam may wear out, and/or may fall apart during the months-long use which is typical for such casts.

Accordingly, the need exists for an improved cast which is compatible with a variety of scanning techniques, which is lighter and more user-friendly, which is both able to be applied at the injury site and to also act as a long-term cast, and/or which is adjustable during use. Furthermore, plaster casts are not re-usable and must be discarded after use, leading to significant waste.

### SUMMARY OF THE INVENTION

The present invention relates to a medical cast including an inflatable air cushion, a foam material within the inflatable air cushion, a closable supporting structure external to the inflatable air cushion, and a sealable air intake connected to the inflatable air cushion. The closable supporting structure is of a size and shape sufficient to enclose a body part. The present invention also relates to a method for using such a medical cast to heal an injury.

Without intending to be limited by theory, it is believed that the invention herein, such as the medical cast, significantly improves the healing process by, for example, reducing healing time and/or by enhancing more complete healing. Furthermore, the present medical cast may be durable, comfortable, sterile, easy to apply, hypo-allergenic, light, re-usable, and/or recyclable. The invention herein may be applied at the location where the injury occurred by, for example a first-responder or may be applied in a hospital by a physician. The invention herein may also provide improved and more durable protection and bone alignment than a mere splint. The invention herein has the flexibility to be either opaque or compatible with a variety of scanning techniques, as desired.

### BRIEF DESCRIPTION OF THE DRAWINGS

FIG. 1 shows a schematic diagram of an embodiment of a device for the fixation and healing of humeral shaft fractures;

FIG. 2 shows an exploded view of an embodiment of an adjusting mechanism;

FIG. 3 shows a schematic diagram of the structure of a one-way valve air cushion;

FIG. 4 shows a partial cut-away view of embodiment of the present invention showing the inflatable air cushion and foam material; and

FIG. 5 shows a partial cut-away view of an embodiment of the present invention for an upper arm;

The figures herein are for illustrative purposes only and are not necessarily drawn to scale.

### DESCRIPTION OF THE PREFERRED EMBODIMENTS

Unless otherwise specifically provided, all tests herein are conducted at standard conditions which include a room and testing temperature of 25° C., sea level (1 atm.) pressure, pH 7, and all measurements are made in metric units. Furthermore, all percentages, ratios, etc. herein are by weight, unless specifically indicated otherwise.

As used herein, the term “external” indicates farther away from the body, the injury, etc.

As used herein, the term “air” indicates any type of gas, including inert gasses, that may be included in the inflatable air cushion. From a practical standpoint, air at ambient temperature is preferred due to its abundance, safety, and ease of use. However, based on the current application, one skilled in the art could envisage employing, for example a gas with a lower-than ambient temperature so as to reduce swelling and cool an inflamed body part, etc.

Unless otherwise indicated, one skilled in the art understands that the materials and mechanisms herein may be obtained, purchased, and/or custom-ordered from a variety of suppliers and vendors worldwide, in a variety of grades and qualities.

### Humeral Shaft Fixation and Healing Device

A device for the fixation and healing of humeral shaft fractures comprises an upper arm fixation mechanism, a forearm fixation mechanism and an adjusting mechanism in connection with both the upper arm fixation mechanism and forearm fixation mechanism. The upper arm fixation mechanism comprising, a first molded base and a first molded cover that matches with said first molded base. The forearm fixation mechanism comprises a second molded base and a second molded cover that matches with said second molded base. The adjusting mechanism is used for adjusting an included angle between the upper arm fixation mechanism and the forearm fixation mechanism so as to fix the positions of said upper arm fixation mechanism and said forearm fixation mechanism.

In one embodiment, the adjusting mechanism comprises a housing, and a rotating member and fixation plates provided in said housing. The housing is connected to said upper arm fixation mechanism, and there is a set of said fixation plates, wherein said fixation plates are connected to said housing, and said rotating member is disposed between two fixation plates. The rotating member is connected with said forearm fixation mechanism. And the center of the housing, the fixation plates and the rotating member is connected through a clevis pin.

In one embodiment, the adjusting mechanism includes two adjusting buttons and one quick lock button. The adjusting buttons are provided on the side of the housing, and the quick lock button is provided on the housing; wherein the adjusting buttons are adapted to adjust the included angle between the upper arm and forearm fixation mechanism, and said quick lock button is adapted to quickly lock the included angle between the said upper arm and forearm fixation mechanism at the positions of 0 °, 15° and 30 °.

In one embodiment, the inside of the upper arm fixation mechanism and the forearm fixation mechanism are equipped with an air cushion. The air cushion may be provided with a plurality of interconnected air chambers, and a foam material may be placed inside each of the air chambers; wherein one end of the air cushion is connected with a one-way valve while the other end is connected to a Velcro tape.

In one embodiment, the air cushion is provided with pockets, the receiving bags are adapted to receive aluminum strips, and said aluminum strip can be bent easily.

In one embodiment, the present fixation and healing device is provided with a hand pump and a pressure gauge.

The structural design of the above embodiment of a humeral shaft fracture fixation and healing device is simple and reasonable. It provides the muscles with uniform closed surrounding pressure through air cushions provided with bendable aluminum strips. The aluminum strips are bent into varying curvatures to fit different patient's needs, thereby correcting angle problems at the junction of the fractures during rehabilitation. Further, the adjustment mechanism can adjust the angle between the upper arm and the forearm fixation mechanism according to the physician's specific requirements so as to provide controllable supports for the humeral shaft fracture and to maintain a fixed axial position that may effectively shorten the healing time of the humeral shaft fracture as well as reduce the risk of improper union of the fractures.

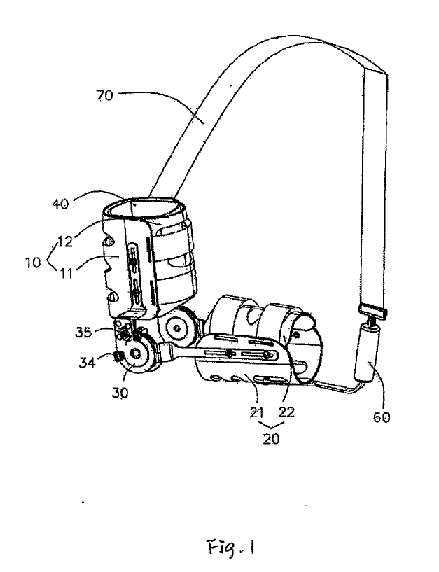

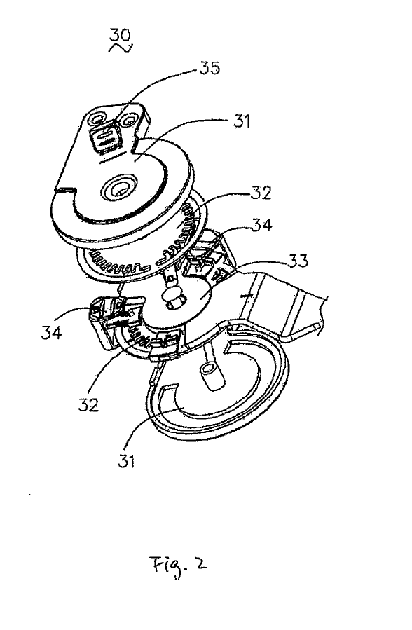

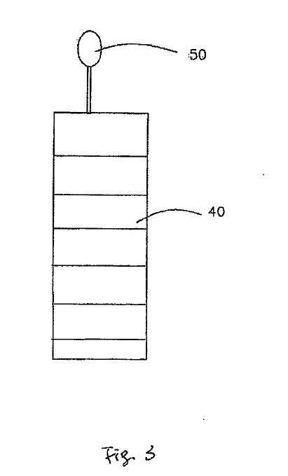

Turning to FIGS. 1-3, a device for the fixation and healing of humeral shaft fractures has an upper arm fixation mechanism 10, a forearm fixation mechanism 20 and an adjusting mechanism 30 in connection with both the upper arm fixation mechanism 10 and the forearm fixation mechanism 20. The adjusting mechanism 30 is used for adjusting an included angle between the upper arm fixation mechanism 10 and the forearm fixation mechanism 20 so as to fix the positions of said the upper arm fixation mechanism 10 and said forearm fixation mechanism 20.

The upper arm fixation mechanism 10 has a first molded base 11 and a first molded cover 12 that matches with said first molded base 11. An air cushion 40 is disposed inside the first molded base 11 and the first molded cover 12. The forearm fixation mechanism comprising, a second molded base 21 and a second molded cover 22 that matches with said second molded base 21. Air cushion 40 is also placed inside the second molded base 21 and the second molded cover 22.

The air cushion 40 is provided with a plurality of interconnected air chambers, and a foam material is placed inside each of the air chambers; wherein all the air chambers are in communication with each other. Moreover, one end of the air cushion 40 is connected with a one-way valve 50 while the other end of the air cushion 40 is connected to a Velcro tape.

The air cushion 40 is provided with pockets (not shown in the drawings), the pockets are adapted to receive aluminum strips (not shown in the drawings), and said aluminum strips can be bent easily. According to the situation of the patient, the aluminum strips can be bent into various curvatures, thereby preventing angulation at fracture site.

The adjusting mechanism 30 has a housing 31, as well as a rotating member 33 and fixation plates 32 provided inside said housing. The housing 31 is connected to said upper arm fixation mechanism 10, and there is a set of said fixation plates 32, wherein said fixation plates 32 are fixedly connected to said housing, and a plurality of slots are provided at the edge of the fixation plates 32. The rotating member 33 is disposed between two fixation plates 32. The rotating member 33 is connected with said forearm fixation mechanism. The center of the housing 31, the fixation plates 32 and the rotating member 33 is connected through a clevis pin. The adjustable angles of the adjusting mechanism are 0-120°.

The adjusting mechanism 30 includes two adjusting buttons 34 and one quick lock button 35. The adjusting buttons 34 are provided on the side of the housing 31, and the quick lock button 35 is provided on the housing; wherein the adjusting buttons 34 are adapted to adjust the included angle between the upper arm fixation mechanism 10 and the forearm fixation mechanism 20, and said quick lock button 35 is adapted to quickly lock the included angle between the said upper arm fixation mechanism 10 and said forearm fixation mechanism 20 at the positions of 0°, 15° and 30°. The adjusting buttons 34 and the quick lock button 35 are adapted to adjust the included angle between the upper arm fixation mechanism 10 and the forearm fixation mechanism 20, said adjusting buttons 34 are connected to said rotating member 33, and a spring (not shown in the drawing) is provided inside the adjusting buttons 34.

A handle 60 is disposed at the end of the forearm fixation mechanism 20, said handle 60 is connected with a shoulder strap 70, wherein the shoulder strap 70 goes through the handle 60 and is connected to the end of the upper arm fixation mechanism 10.

The present fixation and healing device may further include a hand pump and a pressure gauge (not shown).

When the invention is in used; the arm is placed inside the upper arm fixation mechanism 10 and the forearm fixation mechanism 20. The included angle between the upper arm fixation mechanism 10 and the forearm fixation mechanism 20 is adjusted through said adjusting mechanism. To further adjust the angle, it is only necessary to pull out the adjusting button 34 and turn the adjusting button 34 at the same time. The adjusting button will drive the rotation of the rotating member 33 to a required angle. And when the adjusting button is released, the power of the spring will bring the adjusting button back to its original position, which will then lock into the slot at the edge of the fixation plate. In that way, the angle is adjusted and is locked in its desired position. Besides, bendable aluminum strips are equipped inside the air cushion 40 to prevent angulation at the fracture site.

In summary, the structural design of the present invention is simple and reasonable. It provides the muscles with uniform closed surrounding pressure through air cushions equipped with bendable aluminum strips. The aluminum strips can be bent into various curvatures to fit different patient's needs, thereby correcting angle problems (angulation) at the junction of the fractures during rehabilitation. Further, the adjustment mechanism 30 can adjust the angle between the upper arm fixation mechanism 10 and the forearm fixation mechanism 20 according to the specific requirements and provides controllable supports for the humeral shaft fracture so as to maintain a fixed axial position and effectively shorten the healing time of the humeral shaft fracture as well as reduce the risk of improper union of the fractures.

### Medical Cast

An embodiment of the present invention includes a medical cast containing an inflatable air cushion, a foam material within the air cushion, a closable supporting structure, and a sealable air intake. The closable supporting structure is external to the air cushion, and the sealable air intake is connected to the inflatable air cushion. The closable supporting structure has a size and shape sufficient to enclose a body part.

It has now been found that providing a pressure on bones during the healing process provides improved and accelerated healing. The inventors herein recognized that current casts fail to provide such pressure after the muscles atrophy and start to decrease in volume. Accordingly, the present invention provides an apparatus and method for maintaining pressure upon the healing body part. Without intending to be limited by theory, it is believed that the medical cast herein may provide pressure; or a substantially uniform pressure; or a uniform pressure; to the injured body part over the entire healing period, if needed. Specifically, the foam material within the inflatable air cushion provides a certain minimum pressure to the injured body part, while the air pressure in the inflatable air cushion itself may be easily adjusted so as to increase or decrease such pressure, as desired by the physician and/or patient.

As used herein, the term “uniform pressure” indicates that a majority of the portions of the body part enclosed by the closable supporting structure encounter the same pressure. In an embodiment herein the term “uniform pressure” indicates that the % pressure variance is less than or equal to 5% across the area covered by the closable supporting structure. Such pressure may be measured with, for example, a commercially-available flexible flat (sheet-like) pressure monitor as is known in the art such as the X3PRO available from XSENSOR® Technology Corporation.

Such a pressure monitor may be wrapped around the body part so or otherwise inserted so that it is between the body part and the closable supporting structure. Then the inflatable air cushion may be inflated so as to provide a pressure reading. The pressure exerted on the body part may then be represented graphically and the intensity calculated. For example, the pressure monitor is connected to a computer and shows a “map” of the pressure recorded by the flat pressure monitor, and which correlates to the pressure exerted upon the body part at various locations. This allows the average pressure to be calculated by the software, as well as the standard deviation of the pressure. The % pressure variance is calculated by dividing the standard deviation of the pressure by the average pressure.

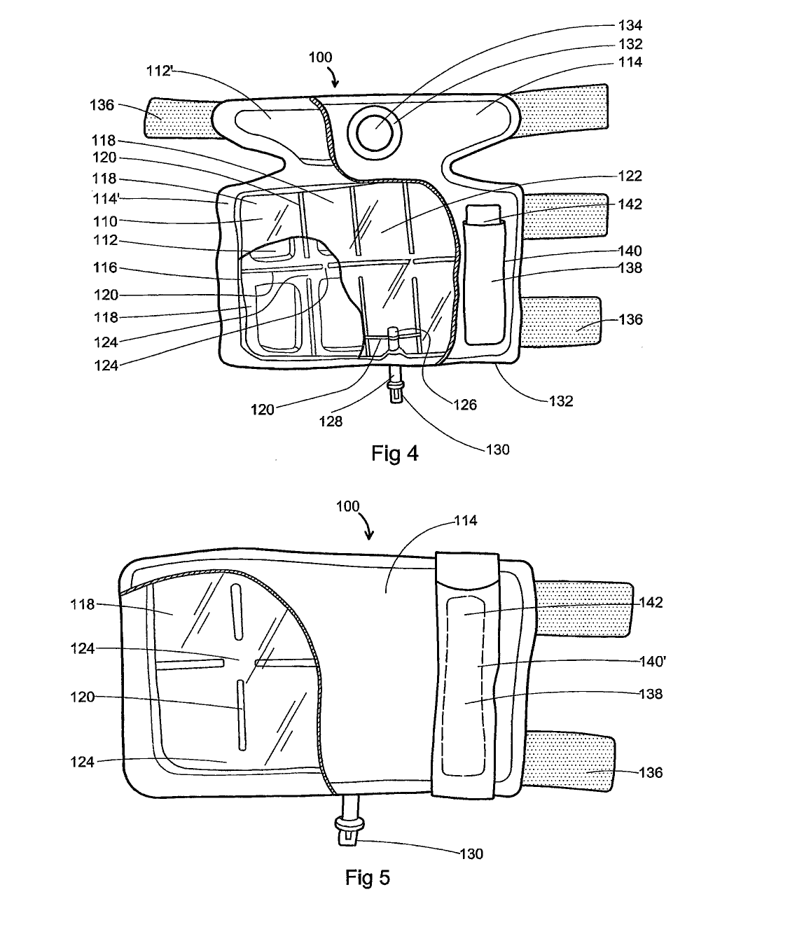

FIG. 4, shows a partial cut-away view of embodiment of the medical cast, 100, of the present invention showing the inflatable air cushion, 110, and a foam material, 112, inside of the inflatable air cushion, 110. In the embodiment of FIG. 4, the inflatable air cushion, 110, is sandwiched between portions of the closeable supporting structure, 114. A portion, 114′, of the closeable supporting structure, 114, is external to the inflatable air cushion, 110, meaning that when the closeable supporting structure is closed around the body part, the portion of the closeable supporting structure is outside of the inflatable air cushion, opposite of the body part which is inside.

In the embodiment of FIG. 4, the inflatable air cushion, 110, is made from a transparent plastic film, for example polyvinyl chloride (PVC), polyethylene, polyurethane, and a combination thereof. In an embodiment herein, the inflatable air cushion is made from polyvinyl chloride or from polyethylene, a thermoplastic polyurethane and may be either opaque, transparent and/or colored as desired. The material for the inflatable air cushion should be flexible, air tight, and resilient to abrasion and temperature changes.

Without intending to be limited by theory, it is believed that it is desirable to exert a pressure; or a substantially uniform pressure; on the injured body part of from about 1 mm Hg (133.3 Pa) to about 80 mm Hg (10,665.8 Pa); or from about 5 mm Hg (666.6 Pa) to about 60 mm Hg (7,999.3 Pa); or from about 8 mm Hg (1,066.8 Pa) to about 50 mm Hg (6,666.1 Pa); or from about 10 mm Hg (1,333.2 Pa) to about 40 mm Hg (5,332.9 Pa) so as to accelerate and/or improve healing of the injured body part, especially the bone. For the purposes herein, we assume the approximation that 1 mm Hg=133.3 Pascals (Pa) at 0° C.

However, it is recognized that too high of a pressure could lead to medical complications and therefore the inflatable air cushion may be designed with a release valve therein so as to release excess pressure. In an embodiment herein, the inflatable air cushion has a burst threshold of greater than or equal to about 100 mm Hg (13,332.2 Pa); or of from about 100 mm Hg (13,332.2 Pa) to about 300 mm Hg (39,996.7 Pa).

The foam material useful herein is generically described as a sponge, or is often more specifically known as a viscoelastic memory foam material and/or a memory foam material. The foam material may be temporarily deformed under pressure and then return to its original shape and volume once the pressure is removed. Such memory foam materials are well known and may include a material selected from polyurethane, polyethylene, ethylene vinyl acetate, latex, rubber, and a combination thereof; or acrylic polyethylene, polyurethane, ethylene vinyl acetate and a combination thereof; or polyurethane, ethylene vinyl acetate and a combination thereof.

In FIG. 4, it can be seen that additional, optional foam material, 112′, which is not within the inflatable air cushion may be included so as to enhance comfort, provide additional pressure, etc.

The medical cast, 100, and specifically the closeable supporting structure, 114, herein is designed to have a size and shape sufficient to enclose a variety of body parts, such as, but not limited to, the extremities and/or where the extremities meet the torso; or the arms and/or legs; or the upper arm, elbow, lower arm, wrist, hand, upper thigh, knee, calf, ankle, and/or foot; or the upper arm, elbow, lower arm, wrist, upper thigh, knee, calf, and/or ankle. In an embodiment herein the medical cast and specifically the closeable supporting structure are intended to accelerate the healing of long bones and therefore is designed to have a size and shape sufficient to enclose the upper arm, elbow, lower arm, wrist, upper thigh, knee, calf, and/or ankle, as it is believed that these bones are especially likely to respond positively to a substantially uniform pressure as described herein.

To exert such a substantially uniform pressure, the inflatable air cushion contains therein the foam material. Without intending to be limited by theory, it is believed that such a structure helps to distribute the air, and therefore the pressure, evenly.

The inflatable air cushion, 110, has a heat-sealed edge, 116, along the perimeter thereof, which ensures that the inflatable air cushion, 110, remains air-tight. Other seals are also useful herein, such as an ultrasonic-sealed edge, an adhesive-sealed edge, a pressure-sealed edge, and a combination thereof; or a heat-sealed edge, an ultrasonic-sealed edge and a combination thereof. Any type of seal is useful herein as long as it helps to maintain the inflatable air cushion air tight.

In an embodiment herein the closable supporting structure includes a material selected from the group consisting of polyurethane, neoprene, a fabric, and a combination thereof; or polyurethane, neoprene, a sandwiched fabric with a TRICOT® outer layer and VELCRO® straps and a combination thereof.

In addition to the heat-sealed edge, 116, the inflatable air cushion, 110, in FIG. 4 contains a plurality of sections, 118, separated by dividers, 120 so as to form a divided inflatable air cushion, 122. The dividers, 120, will typically be formed in the same manner as the edges, and thus in FIG. 4, the dividers are formed by heat-sealing.

For manufacturing simplicity and so as to enable a substantially uniform pressure in all of the sections, 118, the dividers, 120, contain gaps, 124, therebetween so that air may flow between the sections, 118. The inflatable air cushion is therefore a porously-divided air cushion where the gaps, 124, allow air to pass though and help to equalize the pressure between the different sections, 118. Furthermore, in FIG. 4 it can be seen that each section, 118, of the divided inflatable air cushion, 122, contains a foam material, 112, therein.

In an embodiment herein the divided inflatable air cushion contains from about 2 section to about 24 sections; or from about 3 sections to about 20 sections; or from about 4 sections to about 18 sections; or from about 6 to about 15 sections. Without intending to be limited by theory, it is believed that a single section may be difficult to efficiently wrap around a body part, and/or may allow too much movement of the body part. In contrast, too many sections make the air flow more complex and could lead to certain sections being “pinched off” (i.e., where a fold or crease prevents air from circulating between sections) from the rest of the sections. In an embodiment herein, each section is connected to at least one other section by from about 1 gap to about 4 gaps; or from about 2 to about 3 gaps. Without intending to be limited by theory, it is believed that such an arrangement provides an optimum balance between structural stability, structural integrity and avoiding pinched off sections.

In FIG. 4, a sealable air intake, 126, is connected to the inflatable air cushion, 110. The sealable air intake, 126, contains a tube, 128, heat sealed to the inflatable air cushion, 110, at one end, and containing a valve, 130, at the other end. The valve, 130, prevents pressurized air from escaping from the inflatable air cushion, 110, unless it is purposely released. As noted, the air pressure is intended to be maintained at a substantially uniform pressure. In an embodiment herein, the inflatable air cushion contains a single sealable air intake.

The valve, 130, also allows the physician or patient to add air so as to increase the volume of the inflatable air cushion, 110, during use and/or while it is being worn by the patient. Such a feature is especially useful as the body part loses volume during recovery. The ability to add air to the medical cast, 100, during use and/or while it is being worn allows the maintenance of the desired pressure to the body part. The valve useful herein may also contain a pressure release valve for emergency release of air if the pressure gets to high within the inflatable air cushion. Such valves typically contain one or more springs therein calibrated to the desired seal strength and pressures desired.

The tube is typically formed of polyurethane; or a flexible plastic which may be the same or different from that of the inflatable air cushion. The tube may be either clear, opaque, or tinted, as desired.

The closable supporting structure, 114, in FIG. 4 is formed as a hollow bag bounded on the edges by stitching, 132, which seals the inflatable air cushion, 110, and other contents therein. The stitching, 132, may also help to keep the inflatable air cushion, 110, fixed into place, or other methods and structures may be used.

The medical cast, 100, in FIG. 4 is intended for use around the forearm and wrist of a patient, and therefore contains a thumb hole, 134. In other embodiments, the medical cast may include, for example, one or more toe holes, a foot hole, an ankle hole, an elbow hole, etc. to enhance comfort, fit and/or to enhance healing.

The closable supporting structure, 114, also contains a fastener, 136, which in this embodiment includes a hook-and-loop fastener which is used to close the closable supporting structure, 114, around the body part. In an embodiment herein, the fastener is selected from the group consisting of a hook-and-loop fastener, a button, a string, a loop, a hook, a snap, a zipper, a buckle and a combination thereof; or a hook-and-loop fastener, a button, a hook, a snap, a zipper, a buckle and a combination thereof; or a hook-and-loop fastener. In an embodiment herein the fastener is an adjustable fastener. A non-limiting example of a common hook and loop fastener useful herein is VELCRO®, available from the Velcro Company, of Amsterdam, The Netherlands.

The medical cast, 100, also includes a pressure augmenting mechanism, 138, which in this case is shown as a pocket, 140, containing a deformable strip, 142, optionally partially-protruding from therein. The pressure augmenting mechanism is specifically for the purpose of slightly, or not-so-slightly increasing the pressure in specific points so as to, for example, better align the bones so as to improve healing, reduce mis-alignment issues, to align non-union and/or malunion fracture and enhance stability of the fracture location. Such a pressure is considered independently of the pressure exerted by the inflatable air cushions and/or foam material described herein.

The pocket, 140, in FIG. 1 is on the internal side of the closable supporting structure, but this is an optional feature. The pocket useful herein may be either on the internal side of the closable supporting structure, or may be on the external side of the closable supporting structure. In addition, the pocket may be either permanently affixed to the medical cast, or may be removable. In an embodiment herein the pocket is removably-attachable to the medical cast; or the closable supporting structure via, for example, a fastener. Especially, a removably-attachable pocket allows the physician to customize the position of the pocket to put pressure exactly where desired to enhance healing.

The deformable strips useful herein may be formed of any material, and is preferably deformable by hand rather than requiring specific tools. In an embodiment herein the deformable strip is formed from a thermoplastic or aluminium, and a combination thereof; or a plastic, a metal, and a combination thereof; or plastic, aluminium and a combination thereof. In an embodiment herein the material is aluminium. In cases where the physician wants to be able to see the position of the deformable strip relative to the injury, the physician may want to use a deformable strip which is visible to a particular scanning technique—for example, aluminium which is visible on an X-ray. Alternatively, the physician may want to use a deformable strip which is not visible to a particular scanning technique.

In light of the teachings herein, the actual number of pressure augmenting mechanisms useful herein is determinable by the user, or physician. Typically the number of pressure augmenting mechanisms in contact with the closable support structure ranges from about 1 to about 10; or from about 1 to about 8; or from about 2 to about 6. Such pressure augmenting mechanisms may be arranged in series, in parallel, so as to cross, or in any other arrangement as determined by the physician or user.

FIG. 5 shows a partial cut-away view of an embodiment of the medical cast, 100, of the present invention which is sized and shaped for an upper arm or for a thigh. The closeable supporting structure, 114, is shown as viewed from the inside (i.e., this side would be facing the body part). The closeable supporting structure, 114, contains a plurality of sections, 118, separated by dividers, 120. The gaps, 120 between the dividers, 120, allow air to flow between the sections, 118. The valve, 130, is shown as are the fasteners, 136. The pressure augmenting mechanism, 138, is a removably-attachable pocket, 140′, which is wraps around the closeable supporting structure, 114, and is secured on the external side by a fastener (not shown). The removably-attachable pocket, 140′, is movable relative to the closeable supporting structure, 114, and thus may be positioned more to the right or left, as desired. A deformable strip, 142, is enclosed within the pocket, 140.

The medical cast herein may be applied to a patient at the injury site outside of a hospital by, for example, a first-responder, or in a hospital by, for example a physician. Therefore an embodiment of the present invention relates to a method for healing an injury including the steps of applying the medical cast according to Claim 1 to a body part, closing the closable support structure so as to enclose the body part, and inflating the inflatable air cushion with air. In an embodiment herein, the inflatable air cushion may be inflated with, for example, a hand air pump with a pressure gauge, similar to those used to take blood pressure. In an embodiment herein, the inflatable air cushion is inflated with a motorized air pump.

In such a method, the medical cast provides a pressure on the body part which is from about 1 mm Hg to about 80 mm Hg. In an embodiment herein, the pressure is a substantially uniform pressure.

The medical cast herein may be sterilized prior to use, via a variety of methods known in the art. In an embodiment herein, the medical cast herein is provided as a sterilized medical cast; or a sterilized medical cast sealed in a sterile package.

The medical cast herein may be used alone, or in conjunction with the humeral shaft fixation and healing device described herein. For example, the medical cast may be first placed upon the patient, and then the humeral shaft fixation and keeling device described herein, or a corresponding device for a different body part, may be affixed around the medical cast. Alternatively, in an embodiment herein, the medical cast is used without a humeral shaft fixation and keeling device.

Various embodiments of the humeral shaft fractures fixation and healing device are as follows:

1. A humeral shaft fractures fixation and healing device, characterized in that: the fixation and healing device comprising an upper arm fixation mechanism, a forearm fixation mechanism and an adjusting mechanism in connection with both the upper arm fixation mechanism and forearm fixation mechanism. The upper arm fixation mechanism comprising, a first molded base and a first molded cover that matches with said first molded base. The forearm fixation mechanism comprising, a second molded base and a second molded cover that matches with said second molded base. The adjusting mechanism is used for adjusting an included angle between the upper arm fixation mechanism and the forearm fixation mechanism so as to fix the positions of said the upper arm fixation mechanism and said forearm fixation mechanism.
2. The humeral shaft fractures fixation and healing device according to the above item #1, characterized in that: the adjusting mechanism comprises a housing, a rotating member and fixation plates provided in said housing. The housing is connected to said upper arm fixation mechanism, and there is a set of said fixation plates, wherein said fixation plates are connected to said housing and the rotating member is disposed between two fixation plates. The rotating member is connected with said forearm fixation mechanism. And the center of the housing, the fixation plates and the rotating member is connected through a clevis pin.
3. The humeral shaft fractures fixation and healing device according to the above item #1, characterized in that: the adjusting mechanism includes two adjusting buttons and one quick lock button. The adjusting buttons are provided on the side of the housing, and the quick lock button 35 is provided on the housing; wherein the adjusting buttons 34 are adapted to adjust the included angle between the upper arm fixation mechanism and the forearm fixation mechanism. And said quick lock button is adapted to quickly lock the included angle between the said upper arm fixation mechanism and said forearm fixation mechanism at the positions of 0°, 15° and 30°.
4. The humeral shaft fractures fixation and healing device according to the above item #1, characterized in that: the inside of the upper arm fixation mechanism and the forearm fixation mechanism are equipped with an air cushion. The air cushion is provided with a plurality of interconnected air chambers, and a foam material is placed inside each of the air chambers; wherein one end of the air cushion is connected with a one-way valve while the other end of the air cushion is connected to a Velcro tape.
5. The humeral shaft fractures fixation and healing device according to the above item #4, characterized in that: the air cushion is provided with pockets wherein the pockets are adapted to receive aluminum, and said aluminum strips can be bent easily.
6. The humeral shaft fractures fixation and healing device according to the above item #4, characterized in that: the fixation and healing device further includes a hand pump and a pressure gauge.

### EXAMPLE

The pressure provided by a typical plaster cast and a medical brace as described in FIG. 5 were compared. Each was placed around a sample body part and the actual pressure distribution was measured with a commercially-available flat, flexible pressure monitor (pls. give the maker, and model #). A graphical representation of the pressure was created and analyzed by standard computer software to provide the following:

| | Plaster | Medical Cast |
|---|---|---|
| Average contact pressure (mmHg) | 8.8 | 22.0 |
| Standard Deviation (mmHg) | 0.67 | 0.31 |
| % pressure variance across | 7.6% | 1.4% |
| body part | | |
| Sample (n) | 133 | 133 |

Thus, it can be seen that the medical cast provides significantly more average contact pressure and also a lower standard deviation, meaning a substantially uniform pressure across the body part.

It should be understood that the above only illustrates and describes examples whereby the present invention may be carried out, and that modifications and/or alterations may be made thereto without departing from the spirit of the invention.

It should also be understood that certain features of the invention, which are, for clarity, described in the context of separate embodiments, may also be provided in combination in a single embodiment. Conversely, various features of the invention which are, for brevity, described in the context of a single embodiment, may also be provided or separately or in any suitable subcombination.

What is claimed is:

1. A medical cast comprising:
A. an inflatable air cushion;
B. a foam material within the inflatable air cushion;
C. at least a portion of a closable supporting structure external to the air cushion; and
D. a sealable air intake connected to the inflatable air cushion,
wherein the closable supporting structure comprises a size and shape sufficient to enclose a body part.

2. The medical cast according to claim 1, wherein the inflatable air cushion is a divided inflatable air cushion.

3. The medical cast according to claim 1, wherein the foam material is a memory foam material.

4. The medical cast according to claim 1, further comprising a pressure augmenting mechanism.

5. The medical cast according to claim 1, wherein the closable supporting structure further comprises a fastener.

6. The medical cast according to claim 1, wherein the air cushion possess a burst threshold of greater than or equal to about 100 mm Hg.

7. The medical cast according to claim 2, wherein each section of the divided inflatable air cushion comprises a foam material.

8. The medical cast according to claim 4, wherein the pressure augmenting mechanism comprises a pocket and a deformable strip.

9. A method for healing an injury comprising the steps of:
A. applying the medical cast according to claim 1 to a body part;
B. closing the closable support structure so as to enclose the body part; and
C. inflating the inflatable air cushion,
wherein the medical cast provides a pressure on the body part which is from about 1 mm Hg to about 80 mm Hg.

10. The method according to claim 9 wherein the pressure is a substantially uniform pressure.
